(19) United States
(12) Patent Application Publication
(10) Pub. No.: US 2024/0393309 A1
(43) Pub. Date: Nov. 28, 2024

(54) GAS MEASURING DEVICE WITH AUTOMATIC CONFIGURATION DETECTION AND CONFIGURATION PROCESS

(71) Applicant: Dräger Safety AG & Co. KGaA, Lübeck (DE)

(72) Inventors: Matthias MARTENS, Lübeck (DE); Christian WILHELM, Lübeck (DE); Kai EINECKE, Lübeck (DE); Hannes STURM, Lübeck (DE); Florian HANSEN, Lübeck (DE)

(21) Appl. No.: 18/672,335

(22) Filed: May 23, 2024

(30) Foreign Application Priority Data
May 26, 2023 (DE) 10 2023 113 906.1

(51) Int. Cl. G01N 33/00

(52) U.S. Cl. CPC G01N 33/0073

(57) ABSTRACT

An arrangement includes a gas measuring device (100) and an expansion module (20, 21). A process configures the gas measuring device. The gas measuring device includes a base part (1), a gas sensor, an evaluation unit and a reader (17). The gas sensor measures a detection variable that correlates with the concentration of a target gas. The evaluation unit determines the target gas concentration based on a detection variable measured value. The expansion module is detachably connectable to the base part and has an identifier stored in a data memory (19, 24) of the expansion module. With the expansion module connected to the base part, the reader reads the data memory without contacting the expansion module. Depending on a signal from the reader, the gas measuring device determines whether the expansion module is connected to the base part or not and if connected triggers a procedure.

### CROSS REFERENCE TO RELATED APPLICATIONS

This application claims the benefit of priority under 35 U.S.C. § 119 of German Application 10 2023 113 906.1, filed May 26, 2023, the entire contents of which are incorporated herein by reference.

### TECHNICAL FIELD AND BACKGROUND

The invention relates to a configurable arrangement comprising a gas measuring device, wherein the arrangement is capable of automatically recognizing its own current configuration, and to a configuration process for configuring such an arrangement.

### SUMMARY

It is an object of the invention to provide an arrangement comprising a gas measuring device and at least one expansion module, wherein the arrangement can be used in different configurations and wherein the operational reliability of the gas measuring device is increased compared to known arrangements. Furthermore, the invention is based on the task of providing a process for configuring such an arrangement.

The problem is solved by an arrangement with features according to the invention and by a configuration process with features according to the invention. Advantageous embodiments of the arrangement are specified in this disclosure. Advantageous embodiments of the arrangement according to the invention are, where appropriate, also advantageous embodiments of the configuration process according to the invention and vice versa.

The arrangement according to the invention comprises a gas measuring device, which can also be referred to as a gas detection device (gas detector) or analysis device. The gas measuring device is configured to detect at least one predetermined gas in a gas sample, in particular a gas that is combustible and/or toxic to human beings. A gas to be detected is referred to as a “target gas”. The gas sample originates from a spatial area to be monitored, for example from a building or vehicle or an area of a production plant or storage area.

The gas measuring device comprises a base part, at least one gas sensor, a signal-processing evaluation unit, and at least one reader. Preferably, the base part accommodates the gas sensor and a measuring chamber for the gas sample.

The gas sensor is capable of measuring at least one detection variable, in particular an electrical detection variable. The or each detection variable correlates with the respective concentration of the or at least one target gas to be detected. As a rule, the greater the target gas concentration is, the greater is the detection variable, but sometimes the smaller is the detection variable. In one embodiment, the detection variable correlates with the sum of several target gas concentrations.

The evaluation unit is able to determine the concentration of the or at least one target gas to be detected at least approximately. Optionally the evaluation unit is able to determine the summed concentration of several target gases. In order to determine the target gas concentration, the evaluation unit uses at least one measured value of the or at least one detection variable, optionally a time course of the detection variable. In order to determine the target gas concentration, the evaluation unit preferably applies a predefined calculation rule that can be evaluated by a computer to the detection variable.

The arrangement according to the invention comprises at least one expansion module, optionally several expansion modules. The or each expansion module can be detachably connected to the base part, i.e. connected to the base part and also detached again from the base part. The connection is preferably a mechanical connection. If an expansion module is connected to the base part, the possible movement of the expansion module relative to the base part is limited. In one embodiment, the connected expansion module cannot perform any movement at all relative to the base part that is greater than a tolerance. If the expansion module is detached from the base part, the expansion module can generally be moved relative to the base part as desired. In addition to a mechanical connection, the terms “connect to the base part” and “disconnect from the base part” can also include the step of establishing a data connection between the base part and the expansion module or interrupting or terminating this data connection again. It is also possible that the base part supplies a connected expansion module with electrical energy.

In one embodiment, the arrangement comprises several expansion modules. In one embodiment, only one expansion module can be connected to the base part at a time. In another embodiment, several expansion modules can be connected to the base part at the same time (simultaneously).

As already mentioned, the gas measuring device comprises at least one gas sensor. The gas sensor or sensors are capable of measuring a respective detection variable which correlates with a target gas concentration. The or every gas sensor is part of the gas measuring device and is capable of measuring the detection variable, irrespective of whether the or at least one expansion module of the arrangement according to the invention is connected to the base part or the or each expansion module is detached from the base part.

The expansion module or at least one expansion module, preferably each expansion module, comprises a respective expansion module data memory. An identifier (identification) of the expansion module is stored in this expansion module data memory, namely in a form that can be evaluated by a computer. This identifier distinguishes this expansion module from any other item (specimen) or at least from any other type of expansion module that can be detachably connected to the base part. In one embodiment, the identifier distinguishes the expansion module from the or any other expansion module of the arrangement. The identifier comprises, for example, a part number identifying the type of the expansion module and a serial number distinguishing this expansion module from any similar expansion module.

The gas measuring device comprises at least one reader. Preferably, the or each reader is integrated into the base part. The or each reader is capable of reading out the expansion module data memory of an expansion module when the expansion module is connected to the base part. According to the invention, the reader is able to read the expansion module data memory without contact (contactlessly), i.e. to read it even if there is a distance between the reader and the expansion module data memory. The reader is capable of generating a signal, the signal comprising a result of reading out the expansion module data memory. Preferably, the signal comprises either a read-out identification of an expansion module or a code indicating that the reader has not recognized an expansion module or has not been able to read out the data memory and thereby the identification.

The gas measuring device is able to automatically determine whether the or at least one expansion module, which includes an expansion module data memory, is currently connected to the base part or not. For this purpose, the gas measuring device processes the signal from the or at least one reader. It is possible that the gas measuring device comprises several readers and processes the respective signal from each reader.

If the gas measuring device has detected that at least one such expansion module is connected to the base part, the gas measuring device can react as follows:
By evaluating the signal, the gas measuring device is able to determine the stored identifier of an expansion module connected to the base unit.
The gas measuring device automatically triggers at least one procedure. The triggering and/or the type of this procedure depends on the presence and/or the type of identifier stored on the expansion module data memory and determined.

The or a triggered procedure is in particular one of the following procedures:
If the expansion module is present, the gas measuring device provides the concentration of a further target gas, wherein this further target gas cannot be detected with the gas sensor or gas sensors of the gas measuring device. In particular, the expansion module comprises a corresponding additional gas sensor.
If the expansion module is present, the evaluation unit applies a different calculation rule to the detection variable in order to determine the target gas concentration than if the expansion module is absent.
The service life and/or reliability of the gas measuring device is changed, in particular increased, if the expansion module is present. For example, the expansion module is a power supply unit or a cooling unit or a heating unit.
If the expansion module is present, a data connection via radio waves to a remote receiver is set up, and a signal comprising a determined target gas concentration is transmitted to the receiver via this data connection. Preferably, the or one gas sensor of the gas measuring device has measured this target gas concentration. In particular, the expansion module comprises a corresponding communication unit.
In the presence of the expansion module, the gas measuring device provides a result regarding a determined target gas concentration after a modified period of time compared to the absence of the expansion module. For example, when the expansion module is connected to the gas measuring device, a gas sample from the environment needs more time to reach the measuring chamber. Also in this application, the or a gas sensor of the gas measuring device has measured this target gas concentration.
If the expansion module is present, a component of the gas measuring device is enabled (unlocked) and thereby activated. This design makes it possible to install this component in the gas measuring device from the outset, but to activate the component only when the expansion module is present.
If the expansion module is present, the gas measuring device issues an error message because the gas measuring device may not currently be operated with this expansion module. For example, the expansion module or the sensor of the gas measuring device would then be damaged. Or the expansion module interrupts a fluid connection through which a gas sample is to enter a measuring chamber of the gas measuring device when the gas measuring device is used. If the gas measuring device is then operated with the expansion module, there is a risk that a target gas will not be detected.

The configuration process according to the invention relates to such an arrangement and comprises the following steps:
The expansion module or an expansion module is detachably connected to the base part. This expansion module comprises an expansion module data memory in which an identifier of the expansion module is stored.
The reader or a reader of the gas measuring device reads out the expansion module data memory of this expansion module without contact.
The gas measuring device automatically detects that this expansion module is now connected to the base unit. To do this, the gas measuring device uses a signal from the reader.
The gas measuring device determines the identifier stored on the expansion module data memory and read by the reader.
The gas measuring device triggers a procedure depending on the presence and/or type of identifier stored on the expansion module data memory and determined by the gas measuring device.

The invention makes it possible to operate the same base part and thus the same gas sensor of a gas measuring device in conjunction with different expansion modules and thus in different configurations. In particular, the gas measuring device can be adapted to different tasks and/or applications thanks to the different configurations. Thanks to the invention, it is often not necessary to have a separate gas measuring device for each application and/or task. This saves on components.

According to the invention, the or a reader of the gas measuring device reads an identifier that is stored on a data memory of an expansion module. Because the reader reads the identifier without contact, it is in many cases not necessary to position the expansion module exactly in a correct position relative to the reader or to another sensor, for example to a contact switch, of the base part. This feature reduces the risk of a connected expansion module not being detected by mistake or an incorrect expansion module being detected. There is also less risk that the identifier cannot be correctly read/identified due to soiling or damage than with an identifier that can be read in optically.

The gas measuring device is able to react automatically to the read-in identifier and determine that the relevant expansion module is now connected to the base part. Thanks to the invention, it is not necessary for a user to intervene in order to “tell” the gas measuring device that and which expansion module or modules are currently connected to the base part. This reduces the risk of the gas measuring device providing an incorrect measurement result due to a user error. It also saves time in many cases. Conversely, the gas measuring device can automatically determine that it is not currently connected to one or a specific expansion module.

According to the invention, the gas measuring device is able to determine whether the expansion module is connected to the base part or not, depending on the signal from the reader. Preferably, the gas measuring device comprises a program that is stored on a data memory and is executed by a processor during use of the gas measuring device. This program evaluates the or each identifier read in and triggers a reaction of the gas measuring device, this reaction depending on the identifier read in. According to the invention, the or one reader or also an evaluation program determines the identifier of an expansion module, whereby this identifier is stored in the expansion module data memory and the reader reads in this identifier. The gas measuring device automatically evaluates the read-in identifier and reacts to the read-in identifier by carrying out a procedure that depends on the presence and/or type of identifier.

According to the invention, the gas measuring device is able to react automatically to the event that the reader has read the identifier of this expansion module from an expansion module data memory. Different configurations are possible as to how the gas measuring device reacts to this event. Preferably, the reaction to the event depends on which identifier was read in. Particularly preferably, different sets of identifiers are defined, and the reaction depends on which of the predefined sets the read-in identifier belongs to. For example, each possible identifier of an expansion module is or comprises a part number and a serial number, whereby all expansion modules with the same part number are identical in construction and ideally differ from each other by the respective serial number. The reaction of the gas measuring device depends at least on the item number read in, and optionally also on the serial number read in.

According to the invention, the gas sensor measures a detection variable. The evaluation unit uses at least one measured value of the detection variable to determine the target gas concentration. As a rule, the evaluation unit applies a functional relationship between the detection variable and the target gas concentration to the measured detection variable value or detection variable time course in order to derive the target gas concentration. In the simplest case, the functional relationship is a proportionality factor or, more generally, a characteristic curve.

According to the invention, the gas sensor of the gas measuring device is capable of measuring a detection variable. This detection variable correlates with the concentration of at least one target gas. In one embodiment, at least two different functional correlations between the same detection variable and the concentration of the same target gas are stored on an evaluation data memory of the gas measuring device. The evaluation unit or a signal-processing control unit of the gas measuring device automatically selects a functional relationship and applies this to the detection variable value or detection variable curve. If an expansion module of a certain type is connected to the base part, a first functional relationship is applied. If this expansion module is not connected to the base part, a further functional relationship is applied. The two functional relationships differ from each other. The connection of the expansion module to the base part therefore changes a dependency of the detection variable on the target gas concentration. The first correlation is valid and is applied if this expansion module is connected to the base part, the other functional correlation if the expansion module is not connected to the base part.

In a further implementation, three different functional relationships are specified, and the arrangement comprises two different expansion modules. If no expansion module is connected to the base part, a first functional relationship is applied, if the first expansion module is connected, a second functional relationship is applied, and if the second expansion module is connected, a third functional relationship is applied.

Further above, an embodiment of the invention was described in which the evaluation unit applies a first or a second functional relationship to the detection variable, depending on whether an expansion module is connected to the base part or not. In many cases this embodiment makes it possible to take into account and, to a certain extent, compensate for the influence that the expansion module has on the detection variable. One possible application: The expansion module changes the chemical composition and/or a physical property of a gas sample that flows from a spatial area to be monitored into a measuring chamber of the gas measuring device and contains or may contain a target gas. For example, the expansion module absorbs or changes part of a target gas.

In a further development of the embodiment with the two functional relationships, the detection variable depends on the target gas concentration and additionally on an environmental condition, in particular on the ambient temperature and/or the ambient humidity and/or the ambient pressure. This environmental condition occurs in the first functional relationship, while a default value for this environmental condition occurs in the further functional relationship. The or one expansion module comprises a sensor for this environmental condition. It is also possible that the expansion module comprises a receiver, whereby the receiver is configured to receive a signal from a spatially remote sensor for the environmental condition.

If the gas measuring device determines that the expansion module with this environmental condition sensor or this receiver is connected to the base part, the evaluation unit applies the first functional relationship, otherwise the further functional relationship. The gas measuring device uses a signal value from the environmental condition sensor and the first functional relationship to determine a target gas concentration.

In many cases, this configuration makes it possible to take into account the influence of the environmental condition relatively accurately if the expansion module with the sensor measures or receives this environmental condition, and to use a standard (default) value otherwise. Thanks to this advanced implementation with the environmental condition sensor, the same base part can be used both in conjunction with the sensor and without the sensor. It is not necessary for a user to set a default to specify whether or not an environmental condition sensor is connected to the base unit.

In one embodiment, the or an expansion module influences the time it takes for a gas sample from the environment to reach the or a gas sensor of the gas measuring device. This gas sample is taken from an environment of the gas measuring device. In particular, according to this embodiment, the expansion module changes the distance over which a gas sample must flow until it reaches a measuring chamber and thus the or one gas sensor. One possible implementation of this expansion module comprises a hose or a tube or another fluid guide unit that can be connected to the base part in a detachable and fluid-tight manner. This fluid guide unit has two ends, whereby when the fluid guide unit is attached, one end covers an opening of the base part in a fluid-tight manner and the other end is connected to a spatial area that is to be monitored for the presence of a target gas.

If this expansion module is connected to the base part, the following effect is achieved: A gas sample from the spatial area flows through the fluid guide unit and the opening of the base part into the interior of the base part and cannot reach the interior and thus the gas sensor directly, i.e. by bypassing the fluid guide unit. If the expansion module is not connected to the base part, the gas sample can directly flow through the opening into the interior, i.e. without flowing through the fluid guide unit.

Thanks to the fluid guide unit, it is not necessary to install the base part in the spatial area to be monitored. The fluid guide unit, which is connected in a fluid-tight manner, prevents gas from the immediate vicinity of the gas measuring device from reaching the gas sensor. If the fluid guide unit is not connected to the base part, the gas sample from the surroundings passes directly through the opening into the interior of the base part and thus to the gas sensor.

According to this embodiment, the expansion module connected to the base part extends the time that elapses until a gas sample from the spatial area reaches the gas sensor, compared to an embodiment without this expansion module. The evaluation unit takes into account this time duration in the step of determining the target gas concentration as a function of the detection variable, preferably as a function of the time course of the detection variable. In particular, this configuration makes it possible to generate a warning or an alarm regarding the presence of a target gas and/or a message about the absence of the target gas as early as possible, whereby in many cases this configuration ensures that a message that no target gas to be detected is present is only issued when a gas sample from the space to be monitored has reached a measuring chamber inside the base part. As a result, the risk of neither a warning nor an alarm being generated despite the presence of a target gas is relatively low.

According to the invention, the gas measuring device comprises a base part and at least one gas sensor. Preferably, the gas sensor or sensors of the gas measuring device is permanently connected to the base part. It is therefore not necessary to use a reader to check whether a gas sensor of the gas measuring device is connected to the base part or not.

In one embodiment, the or at least one expansion module comprises an additional gas sensor. The additional gas sensor is also capable of measuring a detection variable that correlates with the concentration of a predefined target gas. It is possible that both the detection variable, which the gas sensor of the gas measuring device is able to measure, and the detection variable, which the additional gas sensor of the expansion module is able to measure, relate to the same target gas. In many cases, this increases the reliability that a target gas is actually detected, namely by at least one gas sensor.

An identifier of the additional gas sensor is stored in the data memory of this expansion module and is read in by the reader. Preferably, the evaluation unit is also able to use a detection variable value measured by the other gas sensor, i.e. the gas sensor of the expansion module.

In one embodiment, the additional gas sensor is able to measure a different detection variable than the gas sensor or the gas sensors of the gas measuring device. This increases the range of application of the arrangement. In another embodiment, the additional gas sensor is able to measure the same detection variable as the gas sensor or a gas sensor of the gas measuring device. In many cases, this leads to intentional redundancy, especially if the two gas sensors use different sensor principles. It is also possible that the additional gas sensor can be used in a different environmental condition, e.g. different temperature or humidity, than the gas sensor or gas sensors of the gas measuring device. The additional gas sensor may also have a different service life and/or a different reliability than the gas sensor or gas sensors of the gas measuring device.

The fact that the additional gas sensor is part of an expansion module makes it possible to use the arrangement in one of several possible configurations. In a first configuration, only the gas sensor or sensors of the gas measuring device is used. In a second configuration, the additional gas sensor of the expansion module is additionally used.

It is possible that the arrangement comprises at least two different expansion modules, each with an additional gas sensor. The two additional gas sensors, i.e. the two gas sensors of the two expansion modules, can be configured to measure the same detection variable or to measure two different detection variables.

Preferably, the gas measuring device can be switched on and off. This feature is known to save electrical energy. In one embodiment, the or at least one expansion module comprises a protective element, in particular a sealing cap or closure. If this protective element is connected to the base part, the protective element separates an inner space of the gas measuring device from the environment in a fluid-tight manner. If the protective element is attached, no gas sample can flow from a spatial area to be monitored into the interior of the gas measuring device. In many cases, a connected protective element prevents harmful substances or even a cleaning fluid or water droplets from the environment from entering the interior of the base part and thus reaching a gas sensor in the base part. On the other hand, a connected protective element often prevents a component of a gas sensor from evaporating, which can otherwise happen, in particular with an electrochemical gas sensor.

In one embodiment, this protective element is a component of an expansion module or forms an expansion module. If the gas measuring device is switched on, the invention enables the gas measuring device to automatically determine whether the protective element is connected to the base part or not. For this purpose, the gas measuring device uses a signal from the or a reader, whereby this reader in turn has read in an identifier of the expansion module with the protective element. If the protective element is connected to the base part when the gas measuring device is switched on, the gas measuring device generates an alarm. The gas measuring device emits this alarm in at least one form that can be perceived by a human being or causes an external output unit to emit this alarm in a form that can be perceived by a human being. In particular, the alarm is emitted visually and/or acoustically and/or haptically (through vibrations).

The background: As a rule, the gas measuring device is switched on when it has to analyze a gas sample for at least one target gas. This is only possible if the gas sample reaches a measuring chamber and thus a gas sensor in the interior of the gas measuring device. The gas sample can only reach the measuring chamber if the protective element is removed. Therefore, if the gas measuring device is switched on when the protective element is in place, this is an indication of a fault. If this error is not noticed, there is a risk that a target gas will not be detected. This configuration therefore reduces the risk of the undesirable event of the gas measuring device being operated with the protective element attached to the base part. In many cases, the invention eliminates the need for a human being to check the gas measuring device to see whether the protective element is in place or not. This is often associated with effort, particularly if the gas measuring device is used in a stationary position, i.e. is not carried by a human being. In addition, a user may overlook a protective element that is in place. Furthermore, it is possible, but thanks to the embodiment not necessary, to provide a contact switch that detects the attached protective element.

In one embodiment, the or at least one expansion module comprises a communication module. This communication module can be used to establish a data connection, preferably a data connection via radio waves, between the gas measuring device and a spatially remote receiver. According to the embodiment, this data connection can be established when this expansion module is connected to the base part. Preferably, the base part supplies the connected communication module with electrical energy. The gas measuring device is capable of generating a message and causing the message to be transmitted to the remote receiver via the established data connection. The message comprises an identification of a target gas concentration measured by the gas sensor of the gas measuring device or optionally by another gas sensor of an expansion module, and/or an identification of an alarm generated by the gas measuring device.

The configuration with the communication module as part of an expansion module eliminates the need to permanently equip the gas measuring device with a communication module. Rather, it is possible to use the same gas measuring device on the one hand to warn a user who is carrying the gas measuring device. The gas measuring device then often has an output unit for an alarm. A communication module is then often not required. On the other hand, the gas measuring device can be set up at an installation site and then transmit a message with the target gas concentration to the remote receiver. A communication module is required for this.

In many cases, a gas measuring device is to be used in an environment in which it cannot be connected to a stationary power supply network. Therefore, the gas measuring device preferably comprises its own power supply unit. For example, a user carries the gas measuring device with him/her while he/she are in a spatial area in which at least one target gas can occur. Preferably, the gas measuring device then has an output unit that can warn the user in the event of a high target gas concentration. Or the gas measuring device is set up in a location where a target gas can occur. Preferably, the gas measuring device then comprises a communication unit, and a measured target gas concentration and/or an alarm is transmitted to a remote receiver via this communication unit.

In one embodiment, the or one expansion module comprises an external power supply unit. This external power supply unit can be electrically connected to the gas measuring device. If the expansion module comprising the external power supply unit is connected to the base part, at least one electrical load (consumer) of the gas measuring device is at least temporarily supplied from this external power supply unit.

In some cases, this configuration avoids the need to provide the gas measuring device with an internal power supply unit. Instead, it is possible to connect the gas measuring device selectively to a stationary power supply network or to the external power supply unit. In some cases, this configuration therefore increases the flexibility with which the gas measuring device can be used.

Another embodiment with the external power supply unit is described below. The gas measuring device comprises an internal power supply unit. This is preferably located inside the base part. At least one electrical consumer, preferably each electrical consumer, can be supplied either from the external power supply unit of the expansion module or from the internal power supply unit—the former, of course, only if the expansion module is connected to the base part.

Preferably, a control unit of the gas measuring device has the following effect: If the expansion module comprising the external power supply unit is connected to the base part and the electrical level of the external power supply unit is sufficiently high, the electrical consumers are supplied from the external power supply unit. The internal power supply unit is used if no external power supply unit is connected to the base part or the electrical level of the external power supply unit has fallen below a specified threshold.

In many cases, an external power supply unit can be replaced more quickly than an internal power supply unit. Therefore, the configuration with the external power supply unit makes it possible to quickly connect a new external power supply unit when the internal or the currently connected external power supply unit is low. The replaced old external power supply unit can be recharged while the gas measuring device is being used with the new external power supply unit.

The configuration just described is particularly important in some cases if the gas measuring device is to be used in a potentially explosive environment and is therefore configured as an intrinsically safe device. It is often possible to replace an external power supply unit in the potentially explosive environment. However, the internal power supply unit can only be replaced or recharged outside the potentially explosive environment. The configuration just described therefore avoids the need to carry the gas measuring device out of the potentially explosive environment when a power supply unit is low and to return it to the potentially explosive environment later.

The invention is described below based on embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a front view of a gas measuring device with a display and control unit;

FIG. 2 is a schematic view of an arrangement comprising a gas measuring device with an internal power supply unit and an expansion module with an external power supply unit;

FIG. 3 is a perspective view of the gas measuring device of the embodiment example and a cover cap for the inlets;

FIG. 4 is a perspective view of a part of the gas measuring device in FIG. 3 as well as an adapter and a calibration adapter;

FIG. 5 is a perspective view of the gas measuring device in FIG. 3 and FIG. 4 before fitting the calibration adapter;

FIG. 6 is a perspective view of the gas measuring device in FIG. 5 after fitting the calibration adapter; and

FIG. 7 is an exploded perspective view of a gas measuring device with various expansion modules.

### DESCRIPTION OF PREFERRED EMBODIMENTS

Referring to the drawings, in the various embodiment examples the gas measuring device according to the invention is used to detect at least one harmful target gas in a spatial area, in particular a combustible or toxic target gas, or to ensure that no harmful target gas is present in this spatial area. Optionally, the gas measuring device can detect several harmful target gases at the same time. The spatial area can in particular be a room that is completely or at least partially enclosed and belongs to a building or a vehicle, or it can also be an outdoor area, for example a production plant or storage area.

If several harmful target gases are present in the area, the gas measuring device is preferably able to measure at least the sum of the target gas concentrations. In one embodiment, the gas measuring device comprises several gas sensors and is therefore able to measure the respective concentration of several harmful target gases in the spatial area and/or automatically decide whether the concentration of the or a target gas is below or above a predetermined concentration threshold. In one embodiment, the gas measuring device is capable of outputting at least one measured target gas concentration or an alarm in a form that can be perceived by a human being, i.e. visually and/or acoustically and/or haptically (by vibration). In another embodiment, the gas measuring device is configured to transmit a target gas concentration or an alarm to a remote receiver, which in turn outputs the received message.

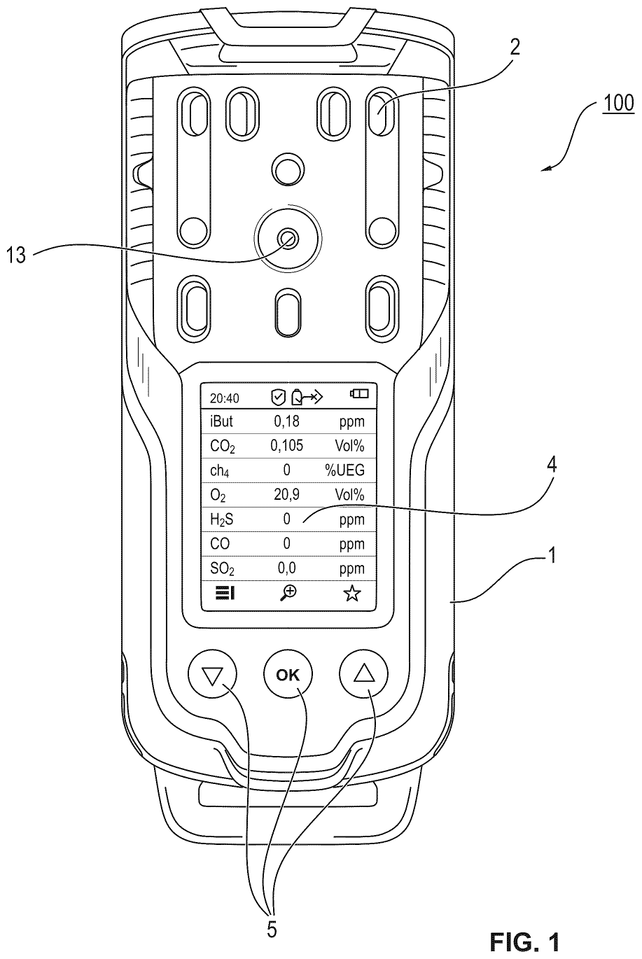

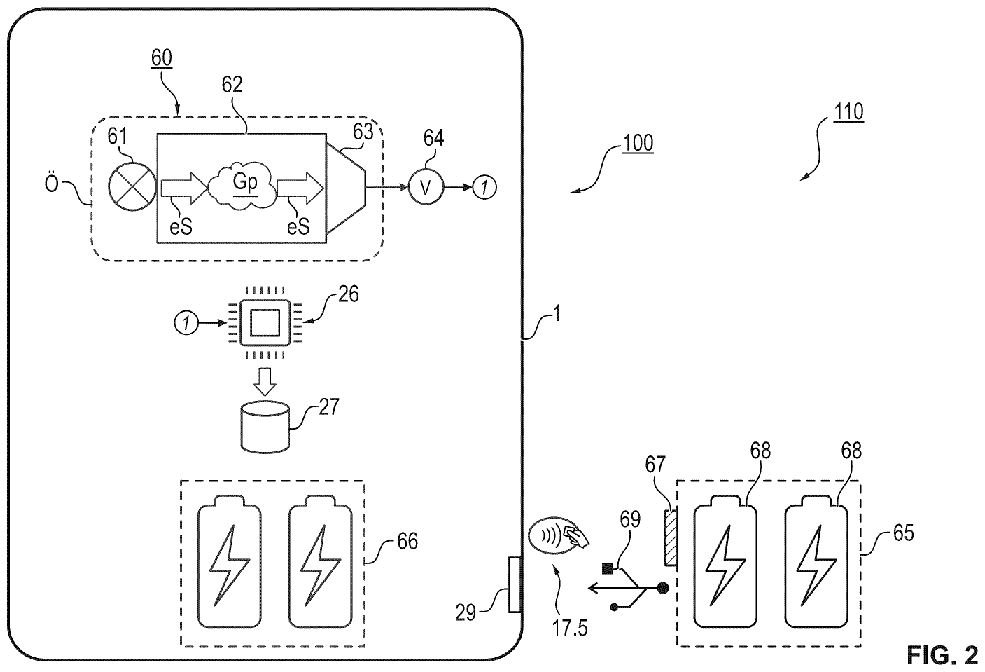

FIG. 1 and FIG. 2 show a perspective view and a schematic view, respectively, of such a gas measuring device 100. The gas measuring device 100 is part of an arrangement 110, wherein the arrangement 110 additionally comprises various expansion modules, which are described below.

A housing 1 encloses a measuring chamber 62 in a fluid-tight manner and accommodates at least one gas sensor, preferably several gas sensors, in its interior. A gas sample Gp to be analyzed flows into the measuring chamber from the outside and reaches the gas sensors there. Preferably, the gas sensors are configured differently so that the gas measuring device 100 is able to detect several combustible target gases in the same gas sample Gp. In addition, redundancy is optionally created by at least two gas sensors being able to detect the same target gas, for example with two different sensor principles.

The gas sensor or sensors of the gas measuring device 100 measure at least one detection variable. The or each detection variable correlates with the presence and/or concentration of a target gas to be detected. The detection variable is, for example, an electrical voltage or a current or an electrical charge or an electrical resistance.

FIG. 2 shows an example of a photoelectric sensor 60. A gas sample Gp to be examined flows from a spatial area to be monitored through an opening Ö into the measuring chamber 62. A radiation source 61 emits electromagnetic radiation eS into the measuring chamber 62. The electromagnetic radiation eS penetrates the measuring chamber 62 and thus the gas sample Gp at least once and strikes a detector 63. A target gas to be detected in the gas sample Gp attenuates the electromagnetic radiation eS. The detector 63 generates a signal, in this case an electrical voltage, which correlates with the intensity of the incident electromagnetic radiation eS. A voltage sensor 64 measures the electrical voltage generated by the detector 63. In the example in FIG. 2, this electrical voltage is the detection variable that correlates with the target gas concentration.

A signal-processing evaluation unit receives the measured value of the or each detection variable from the gas sensor or sensors and derives an estimated value (an indicator) for the target gas concentration from the detection variable value. For this derivation, the evaluation unit applies a predetermined functional relationship to the detection variable that can be evaluated by a computer. The gas measuring device 100 also comprises a signal-processing control unit 26. In the embodiment example, the evaluation unit is a component of the control unit 26. It is also possible that the evaluation unit is spatially remote from the control unit 26 or even from the gas measuring device 100.

The evaluation unit comprises, for example, a program which is stored in a data memory of the control unit 26 and can run on a processor of the control unit 26. At least temporarily, the control unit 26 and thus the evaluation unit have read access to an evaluation data memory 27, in which at least one functional relationship between the detection variable and the target gas concentrations is stored.

Preferably, the gas measuring device 100 comprises its own internal power supply unit 66, which is arranged inside the housing 1 and preferably comprises a plurality of rechargeable batteries (accumulators). This allows a user to carry the gas measuring device 100 while entering an area where a target gas may be present. The gas measuring device 100 can be turned on and off. It is also possible to position the gas measuring device 100 in a stationary position. Thanks to its own power supply unit 66, the gas measuring device 100 does not need to be connected to a stationary power supply network. It is also possible for the gas measuring device 100 to be positioned in a fixed location and connected to a stationary power supply network. In one embodiment, the electrical loads of the gas measuring device 100 can be supplied either from a separate power supply unit or from a stationary power supply network.

In one embodiment, the gas measuring device 100 comprises a socket 29 into which a plug 69 of an expansion module 65 can be inserted. The expansion module 65 further comprises an external power supply unit 68. The electrical loads 26, 61 of the gas measuring device 100 are then supplied with electrical energy by the internal power supply unit 66 if no external power supply unit 68 is connected. If the control unit 26 recognizes that a supplementary module 65 with an external power supply unit 68 is connected, the control unit 26 causes the electrical loads 26, 61 to be supplied by the external power supply unit 68.

In one embodiment, the control unit 26 is able to determine the electrical level of the external power supply unit 68 and compare it with a predetermined lower level threshold. The control unit 26 has the following effect: The electrical loads 26, 61 are only supplied by the external power supply unit 68 if the supplementary module 65 is connected to the gas measuring device 100 and the measured level is above the lower level threshold, and otherwise by the internal power supply unit 66.

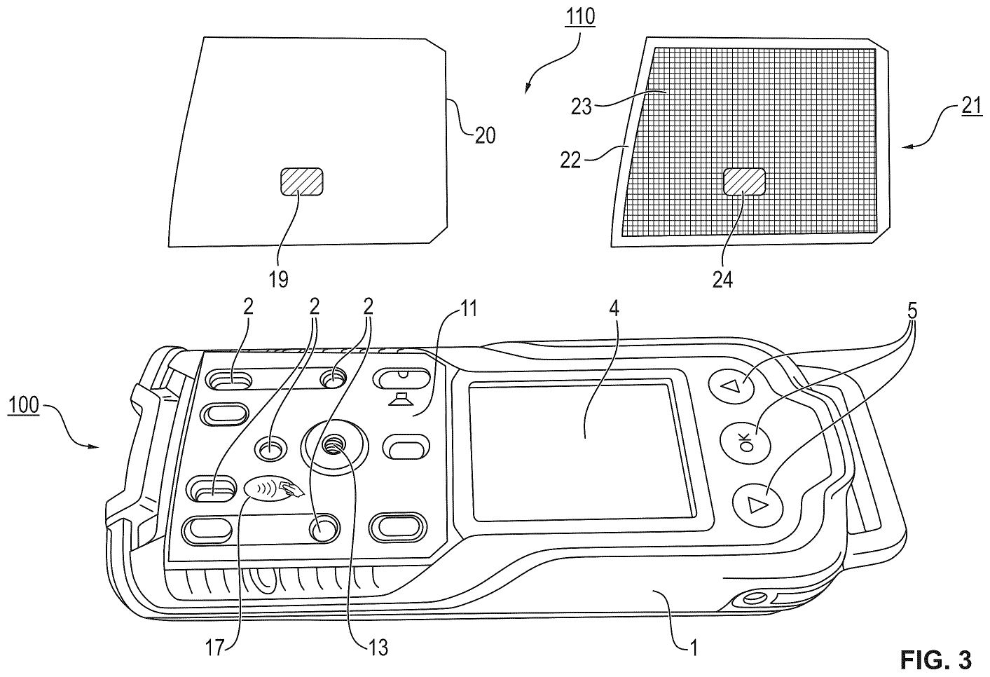

A plurality of inlets 2 are embedded in the housing 1, see FIG. 1 and FIG. 3. The inlets 2 together establish a fluid connection between the environment of the gas measuring device 100 and the gas sensors inside the housing 1, for example the gas sensor 60 of FIG. 2. A display unit 4 and several operating elements 5 are also embedded in the housing 1, see FIG. 1. On the display unit 4, the gas measuring device 100 can visually output measurement results and/or alarms as well as messages about its own status. In one embodiment, the gas measuring device 100 can generate an alarm and output this alarm visually and/or acoustically and/or haptically (by vibrations) if the measured concentration of a target gas or the sum of all target gas concentrations is above a predetermined concentration threshold.

In one application, a fluid diffuses from the surroundings of the gas measuring device 100 through the inlets 2 to the gas sensors inside the housing 1. This application is shown in FIG. 1 and FIG. 3. It is also possible that a pump (not shown) draws in the fluid.

As a rule, the gas measuring device 100 is used intermittently and is temporarily in an idle state, in which it is preferably switched off so that no electrical energy is consumed. It is often desired that the gas measuring device 100 is not in a fluid connection with the environment in the idle state. For this reason, a cover cap 20 can be attached to the inlets 2 in a fluid-tight manner and removed again. FIG. 3 schematically shows the cover cap 20 as well as an approximately rectangular area 11 of the surface of the housing 1, which is covered by the attached cover cap 20. With the cover cap 20 in place, the gas measuring device 100 can be cleaned with a cleaning fluid without the risk of cleaning fluid getting into the interior of the housing 1. Furthermore, no harmful substances or water droplets from the environment can enter the interior of the gas measuring device 100 when the cover cap 20 is in place. A user can place the cover cap 20 on the inlets 2 after a use and remove it again before a new use. Of course, the cover cap 20 must not be in place during use, because then no gas sample can reach the measuring chamber and the gas measuring device 100 cannot detect a target gas.

Furthermore, a grille (grid) unit 21, which is also approximately rectangular, can be placed on the area 11 with the inlets 2. The grille unit 21 reduces the risk of an insect or a larger particle entering the interior of the housing 1 and damaging gas sensors. The grille unit 21 comprises a close-meshed grille 23 and a frame 22.

It is also possible to place a splash guard (not shown) on the area 11 of the inlets 2. This splash guard reduces the risk of liquid droplets entering the interior of the housing 1 during use. Preferably, the cover cap 20, the grille unit 21, the splash guard or none of these three components can be placed on the inlets 2.

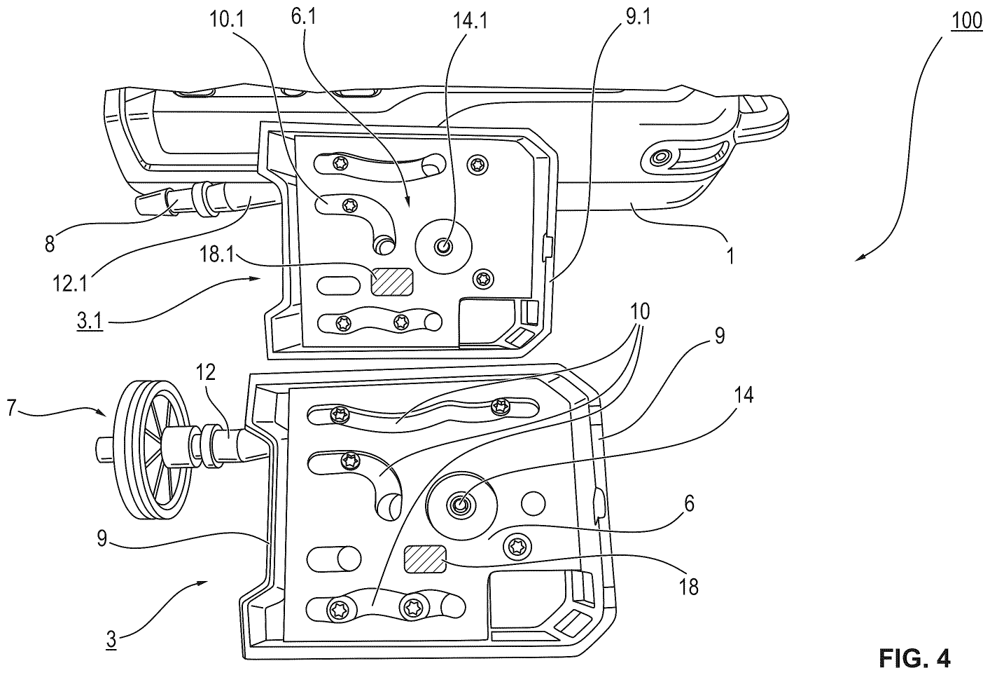

In addition, an adapter 3 can be attached onto the inlets 2 in the housing 1 in a fluid-tight manner, see FIG. 4. If the adapter 3 is attached onto the housing 1, the adapter 3 covers the area 11 and thus interrupts the fluid connection between the inlets 2 and the environment of the gas measuring device 100. Both when the cover cap 20 is attached and when the adapter 3 is attached, there is no fluid connection between the gas sensors in the housing 1 and the environment of the gas measuring device 100 through the inlets 2. FIG. 3 shows a thread 13 for a screw 14, whereby this screw 14 is able to hold the adapter 3 detachably on the housing 1.

The adapter 3 is used during operation of the gas measuring device 100 and comprises
a hard outer shell 9 with a connecting element in the form of a nozzle 12,
an elastic filling 6, which is surrounded by the outer shell 9, and
a screw 14.

FIG. 4 shows the adapter 3 (bottom) and part of the housing 1 (top), with the elastic filling 6 facing the viewer.

With the adapter 3 attached, the screw 14 can be screwed into the thread 13. The filling 6 is then located between the outer shell 9 and the gas sensors. A large number of fluid channels 10 are embedded in the filling 6. These fluid channels 10 establish fluid connections between the nozzle (connection piece) 12 and the gas sensors.

A connecting element 7 can be attached onto the connecting piece 12 in a fluid-tight manner. This connecting element 7 can be connected in a fluid-tight manner to a fluid guide unit, in particular a hose, which is not shown. A fluid delivery unit, for example a pump, can be connected to the fluid guide unit. It is also possible to connect the fluid delivery unit directly to the connecting element 7. In another embodiment, the fluid delivery unit is arranged inside the housing 1.

Thanks to the hose and the fluid delivery unit, the gas measuring device 100 is able to draw in a gas sample to be analyzed. Before being sucked in, the gas sample is located at a measuring position at a distance from the gas measuring device 100. The connecting element 7, the nozzle 12 and the fluid channels 10 pass the sucked in gas sample on to the sensors in the housing 1. It is possible that the gas sample comes from an enclosed space and the gas measuring device 100 is arranged outside this space. This protects the gas measuring device 100 to a certain extent from mechanical and chemical effects that may occur in the enclosed space. The fluid-tight adapter 3 prevents gas from the environment of the gas measuring device 100 from reaching the gas sensors or a gas sample from the interior of the housing 1 from reaching the environment.

The gas measuring device 100 is calibrated before use. A calibration station, not shown, supplies at least one gas sample, preferably several different gas samples, to the gas measuring device 100, the or each gas sample in each case containing a target gas to be detected with a known concentration. In one embodiment, the calibration station comprises at least one compressed gas cylinder, wherein the or each compressed gas cylinder contains a target gas with a known concentration.

During calibration, the value assumed by a detection variable of the gas measuring device 100 for this gas sample is measured in each case. A correlation between the target gas concentration and the detection variable is automatically derived empirically from the measured values and the known concentrations and stored in the evaluation data memory 27 in a form that can be analyzed by a computer. For example, a regression analysis is performed or a neural network is trained.

During calibration, a further adapter 3.1 is placed on the housing 1 instead of the adapter 3, see FIG. 4. To distinguish it from the adapter 3, which is used during operation of the gas measuring device 100, this further adapter 3.1 is called the “calibration adapter”. The outer geometry of the calibration adapter 3.1 matches the outer geometry of the adapter 3, so that either the adapter 3 or the calibration adapter 3.1 can be attached to the housing 1. The attached calibration adapter 3.1 also completely covers the area 11 and prevents a fluid connection to the environment.

The calibration adapter 3.1 includes
a hard outer shell 9.1 with a connection element in the form of a nozzle 12.1,
an elastic filling 6.1 and
one screw 14.1.

FIG. 4 shows the calibration adapter 3.1 in the center, with the filling 6.1 facing the viewer. The screw 14.1 can be screwed into the thread 13.

A large number of fluid channels 10.1 are embedded in the filling 6.1. A fluid-tight calibration connecting element 8 can be attached onto the nozzle 12.1. This calibration connecting element 8 and, optionally, a fluid guide unit (not shown) make it possible to connect the calibration adapter 3.1 to the calibration station in a fluid-tight manner. A gas sample flows from the calibration station through the optional fluid guide unit, the calibration connection element 8 and the fluid channels 10.1 to the gas sensors.

The filling 6 of the adapter 3 and the filling 6.1 of the calibration adapter 3.1 are made of a foam material. This foam is placed in a flowable form in a hollow body, for example pressed, hardens there and is then removed from the hollow body. The filling 6, 6.1 is positively connected to the outer shell 9, 9.1, for example by gluing or screwing. Optionally, at least one sheet is produced from the foam, and several pieces of the filling 6, 6.1 are cut out of this sheet. Cutting out gives each filling 6, 6.1 the desired circumferential contour.

In another embodiment, the foam is applied to the outer shell 9, 9.1, whereby several hollow bodies are held close to the inside of the outer shell 9, 9.1. This causes the fluid channels 10, 10.1 to be formed. In addition, the foam bonds with the outer shell 9, 9.1 during curing.

Both of the manufacturing processes just mentioned make it easier to produce a filling 6, 6.1 with a desired geometry than with any other possible manufacturing process.

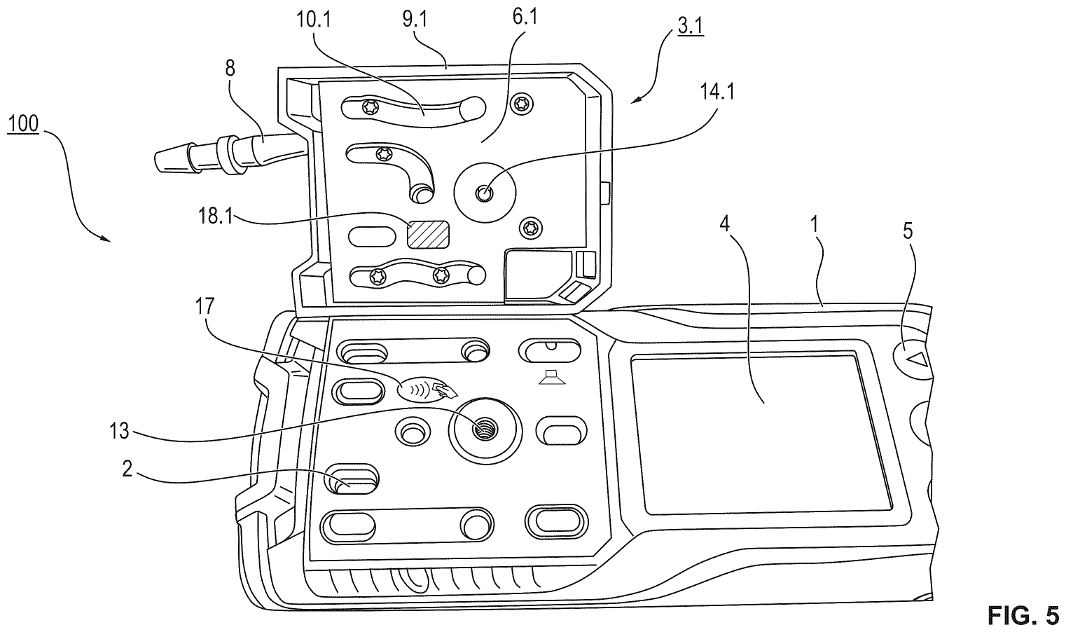

FIG. 5 shows the calibration adapter 3.1 (top) and part of the gas measuring device 100 (bottom) before the calibration adapter 3.1 is placed on the housing 1. The filling 6.1 faces the viewer.

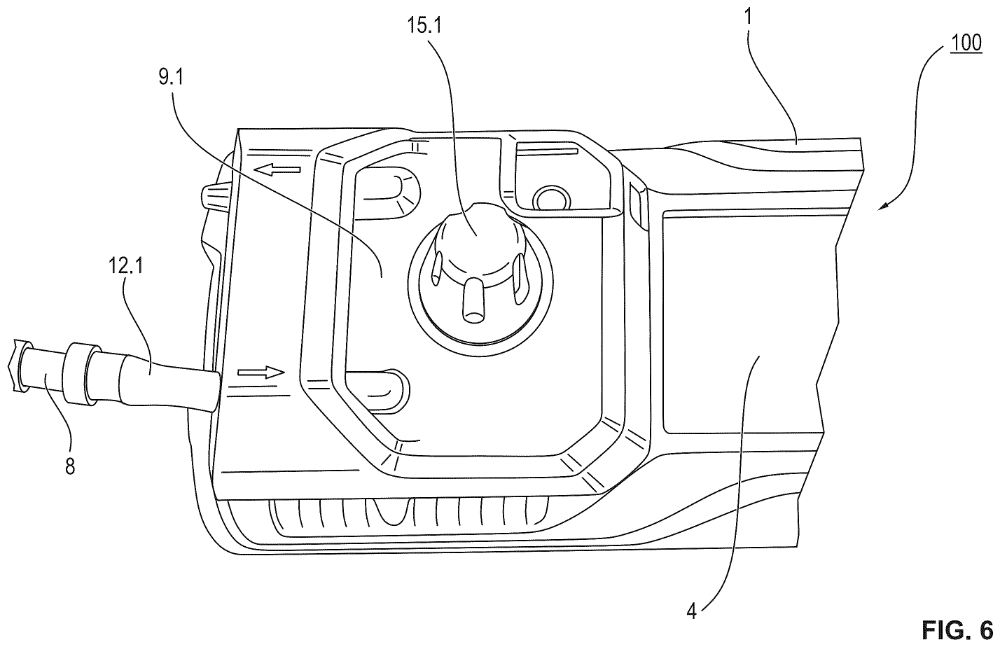

FIG. 6 shows the gas measuring device 100 with the attached calibration adapter 3.1. The calibration adapter 3.1 covers the inlets 2. The outer shell 9.1 faces the viewer. A knob 15.1 is rotatably connected to the outer shell 9.1 and non-rotatably connected to the screw 14.1 and allows the screw 14.1 to be turned from the outside. The connecting piece 12.1 and the calibration connecting element 8 can also be seen.

With reference to the figures, different possible configurations of the gas measuring device 100 have been discussed. The mode of operation of the gas measuring device 100 depends on a current configuration. Some examples are given below.
With the cover cap 20 attached, the gas measuring device 100 is obviously unable to detect any combustible or other target gas. Therefore, the cover cap 20 must be removed when the gas measuring device 100 is used. The fact that the gas measuring device 100 is switched on is an indication that the gas measuring device 100 is now to be used to monitor a spatial area for at least one target gas.
With the adapter 3, 3.1 attached, a gas sample Gp generally takes longer to move from the measuring position into the interior of the housing 1 than if the gas sample Gp passes through the inlets 2 into the interior of the housing 1. Therefore, a reliable measurement result is only available later.
As a rule, the evaluation unit applies a stored functional relationship, for example a proportionality factor, to the measured value of the detection variable in order to determine the concentration of a target gas. It is possible that part of a target gas is absorbed by the elastic filling 6, 6.1 around the fluid channels 10, 10.1 on its way through the fluid channels 10, 10.1 and therefore the elastic filling 6, 6.1 absorbs part of a target gas in the gas sample Gp. Therefore, the detection size (magnitude) depends not only on the target gas concentration, but also on whether the gas measuring device 100 is used with the adapter 3, 3.1 attached or without the adapter 3, 3.1. The evaluation unit therefore applies different functional relationships depending on whether an adapter 3, 3.1 is attached or not.
Even with the grille unit 21 in place, a gas sample often requires more time to reach the gas sensors in the housing 1 compared to a state without the grille unit 21 in place.
The adapter 3 is intended for use of the gas measuring device 100, the calibration adapter 3.1 for calibration. During calibration, the gas measuring device 100 is used differently than during use, namely in a calibration mode instead of a use mode.

If the gas measuring device 100 is connected to the expansion module 65, the electrical loads 26, 61 are supplied by the external power supply unit 68, otherwise by the internal power supply unit 66.

The gas measuring device 100 can therefore be used in different configurations. This configuration can be changed from use to use. According to the invention, the gas measuring device 100 is capable of automatically recognizing its current configuration to a certain extent.

The different configurations described above are due to the fact that an optional expansion module or one of several possible expansion modules can be detachably connected to the housing 1 of the gas measuring device 100. A reader 17, 17.5 is embedded in the housing 1, which is preferably configured as a transceiver (transmitter/receiver) unit. At least one, preferably each expansion module carries a data memory that can be read from the outside. By way of example, FIG. 2 shows a data memory 67 for the expansion module 65 and FIGS. 3 to 5 show a data memory 19 for the cover cap 20, a data memory 24 for the grille unit 21, a data memory 18 for the adapter 3 and a data memory 18.1 for the calibration adapter 3.1.

The reader 17, 17.5 can read any data memory 18, 18.1, 19, 24, 67 without contact, provided that the data memory 18, 18.1, 19, 24, 67 is located in a reading range of the reader 17, 17.5.1, 19, 24, 67 is attached to the expansion module 3, 3.1, 20, 21, 65 in such a way that when the expansion module 3, 3.1, 20, 21, 65 is attached or otherwise connected, the data memory 18, 18.1, 19, 24, 67 is located in the reading area of the reader 17, 17.5. If, on the other hand, the reader 17, 17.5 does not recognize a data memory, the respective expansion module is also not connected to the gas measuring device 100.

The reader 17, 17.5 preferably comprises
a transmitter unit,
an antenna,
a connection to the power supply unit 66, 65 of the gas measuring device 100 or to a stationary power supply network and
its own signal processing unit.

In the embodiment example, each data memory 18, 18.1, 19, 24, 67 is configured as a transponder chip (“tag”), preferably as an RFID chip. The reader 17, 17.5 is configured as an RFID reader. It is also possible that the reader 17, 17.5 uses another suitable process, for example Near Field Communication (NFC), Low Frequency (LF), High Frequency or Ultra High Frequency (UHF).

In an implementation, in order to provide expansion modules with data storage, a sequence of data carriers is applied to a tape, whereby one surface of this tape is connected to an adhesive layer. For example, the tape is an adhesive film. One data memory 18, 18.1, 19, 24, 67 per expansion module is applied to the adhesive layer. A part of the adhesive layer with the data memory 18, 18.1, 19, 24, 67 is adhered to a specific location on the surface of the expansion module 3, 3.1, 20, 21, 65. Preferably, the data memory 18, 18.1, 19, 24 is located between the expansion module 3, 3.1, 20, 21, 65 and the tape, so that the tape protects the data memory 18, 18.1, 19, 24, 67 to a certain extent from environmental influences. As a rule, the tape is permeable to electromagnetic waves.

It is also possible that a housing or another component of the expansion module 3, 3.1, 20, 21, 65 or even the complete expansion module is manufactured by casting. While the component (component or expansion module) is being manufactured, the data memory 18, 18.1, 19, 24, 67 is moved into the interior of this component and temporarily held there. The hardening casting material from which the component is manufactured surrounds the data memory 18, 18.1, 19, 24, 67. The data memory 18, 18.1, 19, 24 is thus brought into the interior of the component by overmolding or by potting.

A data memory 18, 18.1, 19, 24, 67 stores at least the information about the type of expansion module 3, 3.1, 20, 21, 65 to which this data memory 18, 18.1, 19, 24, 67 is applied. It is possible that a unique identifier is also stored, which distinguishes the expansion module from similar expansion modules. Optionally, properties of the expansion module 3, 3.1, 20, 21, 65 are also stored. For example, a proportionality factor between a detection variable that the expansion module is able to measure and a target gas concentration is stored in the data memory, or a time period that elapses until a gas sample has flowed through the expansion module into the interior of the gas measuring device 100.

In the following, several examples are used to describe how the gas measuring device 100 recognizes its own current configuration and uses it automatically during use. The control unit 26 of the gas measuring device 100 receives signals from the or at least one reader 17, 17.5 and thereby information from a read-out data memory 18, 18.1, 19, 24, 67, provided that the corresponding expansion module is set up correctly.
As soon as the control unit 26 has recognized that the expansion module 65 has come into the vicinity of the reader 17.5 and the plug 69 is inserted, the control unit 26 does the following: The electrical loads of the gas measuring device 100 are supplied by the external power supply unit 68. Conversely, as soon as the control unit 26 detects that the expansion module 65 is no longer connected to the gas measuring device 100 or the voltage level (the electrical level) of the external power supply unit 68 has fallen below a predetermined threshold, the control unit 26 switches back to the internal power supply unit 66.
Two advantages of this configuration are: As a rule, the expansion module 65 can be replaced more quickly than the internal power supply unit 66. In some cases, it is also possible to replace the expansion module 65 in a potentially explosive environment, while the internal power supply unit 66 can only be replaced outside this environment.
A generally undesirable situation occurs when the gas measuring device 100 is switched on and the reader 17 recognizes and reads the data memory 19 on the cover cap 20. Based on a corresponding signal from the reader 17, the control unit 26 determines that the cover cap 20 is attached. This is generally undesirable in conjunction with a gas measuring device 100 that is switched on. This is because either a user wants to use the gas measuring device 100 to detect a target gas even though the cover cap 20 is in place, or the user wants to clean the gas measuring device 100 even though it is switched on. Both cases are undesirable and therefore the control unit 26 generates an alarm in both cases. The gas measuring device 100 emits this alarm in a form that can be perceived by a human being, for example visually on the output unit 4 and acoustically.
Various functional relationships between the same detection variable and the concentration of the same target gas are stored in the evaluation data memory 27 of the gas measuring device 100. The evaluation unit has at least temporary read access to the evaluation data memory 27. The current configuration of the gas measuring device 100 determines which of these stored functional relationships the evaluation unit uses for which measured detection variable. The evaluation unit selects a functional relationship depending on a signal from the reader 17. If the gas measuring device 100 comprises several gas sensors in the current configuration, the evaluation unit applies several stored functional relationships.
A first functional relationship is used when the reader 17 has not recognized any data memory of an expansion module. In this case, no expansion module is placed on the area 11 and a gas sample diffuses through the inlets 2 from the environment into the interior of the housing 1.
A second functional relationship is used when the reader 17 has recognized the data memory 18, which shows that the adapter 3 is placed on the area 11. The second functional relationship now used takes into account, on the one hand, that part of a target gas is absorbed by the elastic filling 6. On the other hand, the evaluation unit in the control unit 26 automatically takes into account that a reliable measurement result, i.e. a usable value of the detection variable, is only available after a longer period of time, because the gas sample Gp must pass through a tube and the adapter 3 to the gas sensors in the housing 1 and cannot diffuse directly through the inlets 2, compared to a state without the adapter 3 attached.
In one embodiment, the control unit 26 automatically switches the gas measuring device 100 to a calibration mode when the reader 17 has recognized the data memory 18.1 and has thereby detected that the calibration adapter 3.1 has been placed on the gas measuring device 100. It is not necessary for a user to switch the gas measuring device 100 to calibration mode.
When the reader 17 has detected the data memory 24, it is clear that the grille unit 21 has been placed on the area 11. In this case too, the evaluation unit in the control unit 26 takes into account that a gas sample Gp takes longer to reach the gas sensors. The same can apply to an attached splash guard.

In the embodiment described so far, the gas sensors are fixed inside the housing 1. In a different embodiment, an expansion module, which comprises an external gas sensor, can be detachably connected to the gas measuring device-more precisely: to the base part. This configuration makes it possible to use the same gas measuring device with alternative and/or optional gas sensors.

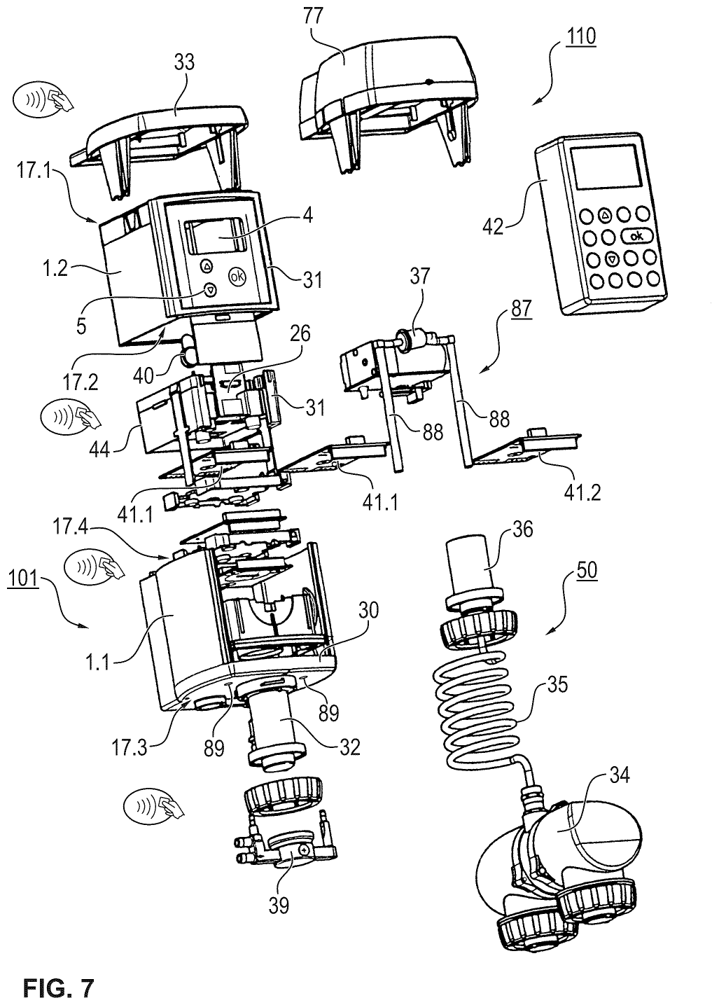

FIG. 7 shows an example of such a gas measuring device 101 in an exploded view. Identical reference signs have the same meanings as in the previous figures. The following can be seen:
a housing unit 30 with a first part 1.1 of the housing 1, two openings 89 in the first part 1.1, a sensor connection 32 and two readers 17.3 and 17.4,
a measuring unit 31 with an internal gas sensor 44, a second part 1.2 of the housing 1, the display unit 4, the operating elements 5, a removable cover 33 and two readers 17.1 and 17.2, wherein the measuring unit 31 also comprises the control unit 26 with the evaluation unit and further electronics (not shown), and
various expansion modules described below.

The internal gas sensor 44 is capable of measuring a detection variable which correlates with the concentration of at least one target gas. The internal gas sensor 44 may be constructed in the same way as the gas sensor 60 described with reference to FIG. 2. The internal gas sensor 44 may also be, for example, a photoacoustic or electrochemical sensor or heat tone sensor. Various expansion modules shown in FIG. 7 each have a data memory that can be read from the outside. These data memories are not shown in FIG. 7.

In one embodiment, the housing unit 30 and the measuring unit 31 are firmly connected to each other. In another embodiment, the two units 30, 31 are detachably connected to each other, for example with the aid of snap fasteners or snap-in fasteners or screw fasteners. If the two units 30, 31 are connected to each other, they form a base part to which various expansion modules can be detachably connected. It is also possible that only the housing unit 30 forms a base part and the measuring unit 31 forms an expansion module. The reader 17.4 of the housing unit 30 is preferably able to read a data memory on the measuring unit 31.

A sensor expansion module 50 with an external gas sensor 34 and a connecting piece 36 can be detachably connected to the sensor connection 32 and optionally screwed on. The gas sensor 34 is also capable of measuring a detection variable that correlates with the concentration of a combustible target gas. In the implementation shown, the connecting piece 36 can be attached and screwed in. If the connecting piece 36 is connected to the sensor connection 32, the gas sensor 34 is supplied with electrical energy via a connecting line 35.

The reader 17.3 is able to read out a data memory on the sensor expansion module 50, wherein this data memory is preferably located on the connecting piece 36. By evaluating a signal from the reader 17.3, the control unit 26 automatically recognizes the type of gas sensor 34 and parameterizes the measuring unit 31 accordingly. It is possible that the internal gas sensor 44 is capable of detecting a first target gas and the external gas sensor 34 is capable of detecting a second target gas.

An adapter 39 for a pump (not shown) can be attached to the sensor connection 32. This pump is able to suck in a gas sample from a remote sampling location. The sucked in gas sample flows, for example, through a hose and the adapter 3 of FIG. 4 to the gas sensor 44. The reader 17.3 is able to read out a data memory on this adapter 39.

If the measuring unit 31 is separate from the housing unit 30, a pump expansion module 87 with a pump 37 can be attached to the housing unit 30 from above. This pump 37 is able to suck in a gas sample and convey it to the gas sensor 44 of the measuring unit 31. When the pump expansion module 87 is attached, two gas lines 88, namely a suction line and a pressure line, lead to the two openings 89 on the underside of the housing unit 30. The delivery direction of the pump 37 can preferably be reversed so that the gas sample can be delivered either to the internal gas sensor 44 of the measuring unit 31 or to the external gas sensor 34 of the connected sensor expansion module 50. The reader 17.4 is able to read out a data memory on the pump expansion module 87.

A release module 40 can be detachably connected to the measuring unit 31. If the release module 40 is connected to the measuring unit 31, the release module 40 releases additional functions of the evaluation unit. The reader 17.2 is able to read a data memory on the release module 40 and thereby recognize whether the release module 40 is connected to the measuring unit 31 or is missing.

In addition, at least one communication module 41.1, 41.2 can be detachably connected to the measuring unit 31. The reader 17.4 is able to read out a data memory on the communication module 41.1, 41.2 in a connected state.

In one implementation, the or at least one communication module 41.1 translates at least one signal from the gas measuring device 101 into a message in accordance with a predetermined communication standard, preferably in accordance with a mobile radio standard. The communication module 41.1 or another communication unit is able to transmit this message to a remote receiver in accordance with the communication standard. This allows the gas measuring device 100 to be read out remotely. A portable device 42 with a display is shown as a receiver. With the aid of this device 42, a user can read out the gas measuring device 101 remotely.

In another implementation, the or at least one communication module 41.2 receives a signal from a gas sensor (not shown) that is capable of measuring a detection variable. The evaluation unit in the control unit 26 activates a corresponding functional relationship between this detection variable and a target gas concentration if such a communication module is detected. This gas sensor can be arranged at a distance from the gas measuring device 101. The measuring unit 31 of the gas measuring device 101 can evaluate a signal from the remote gas sensor. These two forms of implementation can be combined with each other.

A data memory is preferably arranged on the underside of the cover 33. The reader 17.1 is able to read this data memory on the cover 33. If the reader 17.1 does not detect a data memory, an alarm is preferably generated and output. This is because the absence of a cover 33 is an undesirable situation.

The cover 33 can be replaced by an expansion module 77. The expansion module 77 performs the mechanical function of the cover 33 and also contains an optional device, for example a temperature sensor or a humidity sensor or a pressure sensor or a sensor for another environmental condition. The reader 17.1 is able to read a data memory on the underside of the expansion module 77. The evaluation unit in the control unit 26 activates a further functional relationship in the above-mentioned data memory for functional relationships. This is because if the ambient temperature or the ambient humidity or the ambient pressure or the other environmental condition is measured, a functional relationship can be applied in which the temperature or the humidity occurs as a measured variable. If such an expansion module 77 is not detected, a predetermined or user-set default value for the environmental condition is used.

Preferably, the control unit 26 determines the current configuration of the gas measuring device 100, i.e. determines which expansion modules are currently connected to the actual gas measuring device 100 according to the signals from the readers 17, 17.1, . . . , 17.4. A maintenance technician can read out the current configuration of the gas measuring device 100. Preferably, the maintenance technician authorizes himself/herself with the aid of a data memory on a personal card by holding this personal card to a reader 17, 17.1, . . . , 17.4.

While specific embodiments of the invention have been shown and described in detail to illustrate the application of the principles of the invention, it will be understood that the invention may be embodied otherwise without departing from such principles.

### LIST OF REFERENCE SYMBOLS

| | |
|---|---|
| 1 | Housing of the gas measuring device 100, accommodates the display unit 4 |
| | and the operating elements 5, surrounds the gas sensor 60, has the inlets 2 |
| | and the thread 13 |
| 2 | Inlets in the housing 1, each establish a fluid connection between a sensor |
| | of the gas measuring device 100 and the environment, are arranged in the |
| | area 11 of the housing 1 |
| 3 | Adapter, comprises the outer shell 9, the filling 6 and the nozzle 12, can be |
| | detachably attached to the housing 1 and then covers the area 11, is used |
| | when the gas measuring device 100 is used |
| 3.1 | Calibration adapter, comprises the outer shell 9.1, the filling 6.1 and the |
| | nozzle 12.1, can be detachably attached to the housing 1 and then covers |
| | the area 11, is used when calibrating the gas measuring device 100 |
| 4 | Display unit in housing 1 and in measuring unit 31, displays measurement |
| | results |
| 5 | Operating elements in the housing 1 and in the measuring unit 31 |
| 6 | Filling of the adapter 3, is made of a foam material |
| 6.1 | Filling of the calibration adapter 3.1, is made of a foam material |
| 7 | Connecting element that can be attached to the connecting piece 12 and |
| | connected to a hose |
| 8 | Connecting element that can be placed on the connecting piece 12.1 and |
| | connected to a calibration station |
| 9 | Outer shell of the adapter 3 |
| 9.1 | Outer shell of the adapter 3.1 |
| 10 | Fluid channels in the filling 6 |
| 10.1 | Fluid channels in the filling 6.1 |
| 11 | Area in the housing 1 above the inlets 2, which is covered by the attached |
| | adapter 3, 3.1 and the attached cover cap 20 |
| 12 | Nozzle (connection piece) on the outer shell 9 |
| 12.1 | Nozzle (connection piece) on the outer shell 9.1 |
| 13 | Thread in the housing 1 into which the screw 14, 14.1 can be screwed |
| 14 | Screw of the adapter 3, can be screwed into the thread 13 |
| 14.1 | Screw of the calibration adapter 3.1, can be screwed into the thread 13 |
| 15.1 | Knob for the screw 14.1 |
| 17 | RFID reader in housing 1 |
| 17.1, 17.2 | RFID readers on the measuring unit 31 |
| 17.3, 17.4 | RFID readers on the housing unit 30 |
| 17.5 | RFID reader in housing 1, detects the expansion module 65 with the |
| | external power supply unit 68 |
| 18 | RFID chip on the adapter 3 |
| 18.1 | RFID chip on the calibration adapter 3.1 |
| 19 | RFID chip on the cover cap 20 |
| 20 | Cover cap for the inlets 2, covers the area 11, encloses the RFID chip 19 |
| 21 | Grille (grid) unit, comprising the grille 23, the frame 22 and the RFID chip |
| | 24, can be placed on the inlets 2, then covers the area 11 |
| 22 | Frame for the grille 22 |
| 23 | Close-meshed grille (grid) of the grille unit 21 |
| 24 | RFID chip on the grille unit 21 |
| 26 | Signal-processing control unit with an evaluation unit, the control unit |
| | recognizing the current configuration of the gas measuring device 100, 101 |
| | and the evaluation unit determining a target gas concentration |
| 27 | Evaluation data memory to which the evaluation unit has read access |
| 29 | Socket in housing 1, can accommodate plug 69 |
| 30 | Housing unit, comprises the part 1.1 of the housing 1, the sensor |
| | connection 32, the openings 89 and the RFID readers 17.3 and 17.4 |
| 31 | Measuring unit, comprises part 1.2 of housing 1, gas sensor 44 and RFID |
| | readers 17.1 and 17.2 |
| 32 | Sensor connection of the housing part 30 |
| 33 | Cover, can be placed on the measuring unit 31 from above |
| 34 | Gas sensor, belongs to sensor expansion module 50 |
| 35 | Electrical connection cable for the gas sensor 34, belongs to the sensor |
| | expansion module 50 |
| 36 | Adapter of the sensor expansion module 50, can be attached to the sensor |
| | connection 32 |
| 37 | Pump, belongs to pump expansion module 87 |
| 39 | Adapter for a pump not shown, can be attached to sensor connection 32 |
| 40 | Release module, releases functions of the evaluation unit |
| 41.1 | Communication module, is configured to translate signals from gas sensors |
| | of the gas measuring device 101 into a message according to a |
| | communication standard and cause the message to be transmitted to a |
| | spatially remote receiver |
| 41.2 | Communication module, is configured to receive a signal from a remote |
| | gas sensor and forward it to the measuring unit 31 |
| 42 | Spatially remote device, is configured to receive signals from gas sensors |
| | of the gas measuring device 101 and output them in at least one form |
| | perceptible by a human being |
| 44 | Gas sensor of the measuring unit 31 |
| 50 | Sensor expansion module, comprises the gas sensor 34, the connection line |
| | 35 and the adapter 36, can be detachably connected to the sensor |
| | connection 32 |
| 60 | Photo-electric gas sensor of the gas measuring device 100 |
| 61 | Radiation source of the gas sensor 60 |
| 62 | Measuring chamber of the gas sensor 60 |
| 63 | Detector of the gas sensor 60 |
| 64 | Voltage sensor of the gas sensor 60, measures the electrical voltage of the |
| | detector 63 as the detection variable |
| 65 | Expansion module with the external power supply unit 68, the connector 69 |
| | and the data memory 67 |
| 66 | Internal power supply unit of the gas measuring device 100 |
| 67 | Data memory of the expansion module 65 |
| 68 | Power supply unit of the expansion module 65 |
| 69 | Connector of the expansion module 65 |
| 77 | In one embodiment, the expansion module, which can replace the cover 33, |
| | comprises a sensor for an environmental condition |
| 87 | Pump expansion module, includes the pump 37 and the lines 88, can be |
| | attached to the measuring unit 31 from above |
| 88 | Lines of the pump expansion module 87, App can be fed through the |
| | housing unit 30 and then protrude out of the openings 89 |
| 89 | Openings in the housing unit 30 through which the cables 88 can be routed |
| 100 | Gas measuring device, comprises the housing 1, the adapter 3, 3.1, the |
| | display unit 4, the operating elements 5 and the RFID reader 17 as well as |
| | gas sensors not shown |
| 101 | Gas measuring device, comprises the housing unit 30, the measuring unit |
| | 31 and the RFID readers 17.1, . . . , 17.4 |
| 110 | Arrangement, comprises a gas measuring device 100, 101 and various |
| | expansion modules 3, 3.1, 20, 21, 36, 40, 41.1, 41.2, 50, 77 |
| eS | Electromagnetic radiation, simulated by the radiation source 61, penetrates |
| | the measuring chamber 62 and the gas sample Gp, impinges upon the |
| | detector 64 |
| Gp | Gas sample in the measuring chamber 62 |
| Ö | Opening in the measuring chamber 62, through which the gas sample Gp |
| | enters the measuring chamber 62 |

What is claimed is:

1. An arrangement comprising:
a gas measuring device, the gas measuring device comprising:
a base part;
a gas sensor configured to measure a respective value of at least one detection variable which correlates with a concentration of at least one target gas;
a signal-processing evaluation unit configured to evaluate the measured value for the detection variable and to determine a target gas concentration or a summed concentration of target gases based on the evaluation of the measured value; and
at least one reader; and
at least one expansion module configured to be detachably connected to the base part, the expansion module comprising an expansion module data memory in which an identifier of the expansion module is stored,
wherein the reader is configured to contactlessly read out the expansion module data memory in a state with the expansion module connected to the base part,
wherein the gas measuring device is configured to determine, depending on a signal from the reader, whether or not the expansion module is connected to the base part and, upon determining the expansion module is connected to the base part, to determine the stored identifier and, depending on a presence of the stored identifier and/or a type of the stored identifier, to trigger a procedure.

2. An arrangement according to claim 1, further comprising an evaluation data memory,
wherein the expansion module is configured to change a dependence of the detection variable on the target gas concentration and/or to influence the determination of the target gas concentration as a function of the detection variable,
wherein a first functional relationship between the detection variable and the target gas concentration is stored on the evaluation data memory and a further functional relationship between the detection variable and the target gas concentration is stored on the evaluation data memory,
wherein the evaluation unit has at least temporary read access to the evaluation data memory,
wherein the evaluation unit is configured to apply the first functional relationship to the measured value of the detection variable in the state with the expansion module connected to the base part, and
wherein the evaluation unit is configured to apply the further functional relationship to the measured value of the detection variable in a state with the expansion module not connected to the base part.

3. An arrangement according to claim 2, wherein the expansion module comprises a sensor for an environmental condition and wherein the environmental condition occurs in the first functional relationship and a default value for the environmental condition occurs in the further functional relationship.

4. An arrangement according to claim 1,
wherein the arrangement is configured such that in a state with the expansion module connected to the base part a time period for a gas sample from an environment of the gas measuring device to reach the gas sensor of the gas measuring device is extended compared to a state with the expansion module not connected to the base part, and
wherein the evaluation unit is configured to take into account the extended time period for evaluating the measured detection variable value.

5. An arrangement according to claim 4,
wherein an interior of the base part comprises a measuring chamber, the chamber configured to receive a gas sample,
wherein the base part comprises a housing and the housing comprises an opening, the opening being in fluid communication with the measuring chamber,
wherein the expansion module, which extends the time period, comprises a cover and a fluid guide unit, the fluid guide unit being connected to the cover in a fluid-tight manner,
wherein in the state with the expansion module connected to the base part, the cover fluid-tightly covers the opening in the housing such that the gas sample from an environment of the gas measuring device only reaches the measuring chamber through the fluid guide unit of the expansion module and through the opening, and
wherein in the state with the expansion module not connected to the base part, the gas sample reaches the measuring chamber through the opening without flowing through the fluid guide unit.

6. An arrangement according to claim 1, wherein the expansion module comprises an additional gas sensor that is configured to measure a detection variable which correlates with a concentration of a predetermined target gas.

7. An arrangement according to claim 1,
wherein the gas measuring device is configured to be switched on and off,
wherein the expansion module comprises a protective element, the protective element being connected to the base part fluid-tightly separates an inner space of the gas measuring device from an environment of the gas measuring device, and
wherein the switched-on gas measuring device is configured, in response to a signal from the reader, to determine whether or not the protective element is connected to the base part and to generate an alarm in a state with the protective element connected to the base part and to cause the alarm to be issued in a form that can be perceived by a human being.

8. An arrangement according to claim 1,
wherein the expansion module comprises a communication module, and
wherein in a state with the expansion module connected to the base part, the gas measuring device is configured to generate a message comprising at least one determined target gas concentration, to establish, with the communication module, a data connection to a spatially remote receiver, and to cause the message to be transmitted to the receiver via the established data connection.

9. An arrangement according to claim 1,
wherein the expansion module comprises an external power supply unit, and
wherein in a state with the expansion module connected to the base part, the gas measuring device is configured to supply at least one electrical consumer of the gas measuring device from the external power supply unit.

10. An arrangement according to claim 9,
wherein the gas measuring device further comprises an internal power supply unit, and
wherein in the state with the expansion module connected to the base part, the gas measuring device is configured to supply the electrical consumer from the external power supply unit and otherwise from the internal power supply unit.

11. A process for configuring a gas measuring device, the process comprising the steps of:
providing a gas measuring device comprising: a base part; a gas sensor configured to measure a respective value of at least one detection variable which correlates with the concentration of at least one target gas; a signal-processing evaluation unit configured to evaluate the measured value for the detection variable to determine a target gas concentration or a summed concentration of target gases based on the evaluation of the measured value; and a reader;
providing at least one expansion module that is configured to be detachably connected to the base part and comprises an expansion module data memory on which an identifier of the expansion module is stored;
detachably connecting the expansion module to the base part;
with the reader, contactlessly reading out the expansion module data memory of the expansion module;
depending on a signal from the reader, detecting that the expansion module is connected to the base part;
determining the identifier stored in the expansion module data memory; and
depending on a presence of the identifier and/or depending on a type of the identifier, triggering a procedure.
